(19) United States
(12) Patent Application Publication
(10) Pub. No.: US 2017/0349618 A1
(43) Pub. Date: Dec. 7, 2017

(54) DISACCHARIDE SYNTHETIC LIPID COMPOUNDS AND USES THEREOF

(71) Applicants: AVANTI POLAR LIPIDS, INC., Alabaster, AL (US); AC IMMUNE SA, Lausanne (CH)

(72) Inventors: Walter A. SHAW, Birmingham, AL (US); Stephen W. BURGESS, Chelsea, AL (US); Shengrong LI, Birmingham, AL (US); David T. HICKMAN, Saint-Sulpice (CH); Maria Pilar LOPEZ-DEBER, Lausanne (CH)

(21) Appl. No.: 15/175,843

(22) Filed: Jun. 7, 2016

(51) Int. Cl. C07H 13/06; A61K 9/127; A61K 39/39; A61K 39/00

(52) U.S. Cl. CPC C07H 13/06; A61K 39/39; A61K 9/127; A61K 2039/55572; A61K 2039/55555

(57) ABSTRACT

Essentially pure compounds of the formulas (I) to (V) are provided. Compositions and methods for enhancing or stimulating an immune response are also provided. The compounds, provided are advantageous in that the compounds are essentially pure and free from contaminants encountered when such compounds are purified from natural sources.

### FIELD OF THE DISCLOSURE

The present disclosure relates generally to compounds for use in enhancing or stimulating an immune response. More specifically, the present disclosure relates to synthetic disaccharide lipid A analogues having the structure of formulas (I)-(V), or pharmaceutically acceptable salts thereof. The use of such compounds for inducing and stimulating an immune response, pharmaceutical compositions containing such compounds and vaccine compositions comprising such compounds are also disclosed.

### BACKGROUND

It is known that endotoxin, which is an outer membrane component of various Gram negative bacilli, has various biological activities, including enhancement of immune function. The active portion of the endotoxin resides in a disaccharide moiety referred to as lipid A. Various lipid A derivatives have been studied and reported to have biological activities similar to natural lipid A. However, many such compounds are purified from natural sources which can lead to problems with consistency and purity of these compounds. For example, compounds purified from natural sources may result in lipid A with a mixture of acylation patters (for example, lipid A structures having 3-7 acyl chains attached to the disaccharide structure) or a mixture of different acyl chain lengths present at a given position. The impurity of these naturally purified lipid A compounds is disadvantageous when such naturally purified lipid A compounds are to be administered to subject, particularly human subjects. Therefore, much research has centered on providing pure or essentially pure forms of such compounds.

In addition, it is known that the acylation pattern of lipid A molecules can impact the properties of the lipid A molecule. Such properties include, but are not limited to, the immunostimulatory properties. For example, lipid A molecules with 6 acyl chains generally have improved immunostimulatory properties as compared to lipid A molecules with 3 acyl chains. Furthermore, the location of the acyl chains on the lipid A molecule may also impact the properties of the lipid A molecule. Likewise the length of the acyl chains present at a given position may also impact the properties of the lipid A molecule. For example, an acyl chain of 4 carbons in length is generally not as effective as an acyl chain of 12 carbons in length. While the art is aware of a number of lipid A molecules, some of which are available in a synthetic form, there is a need to develop additional lipid A molecules having properties beneficial properties. The present disclosure provides a new series of lipid A molecules in furtherance of this objective.

### DETAILED DESCRIPTION

### Definitions

As used herein, the terms “prevention”, “prevent”, “preventing”, “suppression”, “suppress” and “suppressing” as used herein refer to a course of action (such as administering a compound or pharmaceutical composition) initiated prior to the onset of a symptom, aspect, or characteristics of a disease or condition so as to prevent or reduce such symptom, aspect, or characteristics. Such preventing and suppressing need not be absolute to be useful.

As used herein, the terms “treatment”, “treat” and “treating” as used herein refers a course of action (such as administering a compound or pharmaceutical composition) initiated after the onset of a symptom, aspect, or characteristics of a disease or condition so as to eliminate or reduce such symptom, aspect, or characteristics. Such treating need not be absolute to be useful.

As used herein, the term “in need of treatment” as used herein refers to a judgment made by a caregiver that a patient requires or will benefit from treatment. This judgment is made based on a variety of factors that are in the realm of a caregiver's expertise, but that includes the knowledge that the patient is ill, or will be ill, as the result of a disease or condition that is treatable by a method or compound of the disclosure.

As used herein, the term “in need of prevention/suppression” as used herein refers to a judgment made by a caregiver that a patient requires or will benefit from prevention. This judgment is made based on a variety of factors that are in the realm of a caregiver's expertise, but that includes the knowledge that the patient will be ill or may become ill, as the result of a disease or condition that is preventable by a method or compound of the disclosure.

As used herein, the terms “individual”, “subject” or “patient” as used herein refers to any animal, including mammals, such as mice, rats, other rodents, rabbits, dogs, cats, swine, cattle, sheep, horses, or primates, and humans. The term may specify male or female or both, or exclude male or female. The subject in certain embodiments is a human.

As used herein, the term “therapeutically effective amount” as used herein refers to an amount of a compound, either alone or as a part of a pharmaceutical composition, that is capable of having any detectable, positive effect on any symptom, aspect, or characteristics of a disease or condition. Such effect need not be absolute to be beneficial.

As used herein, the term “alkyl”, whether used alone or as part of a substituent or linking group, includes straight hydrocarbon groups comprising from one to twenty carbon atoms. Thus the phrase includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃), —C(CH₂CH₃)₃, —CH₂CH(CH₃)₂, —CH₂CH(CH₃)(CH₂CH₃), —CH₂CH(CH₂CH₃)₂, —CHC(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃)—, CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)CH(CH₃)₂, —CH(CH₂CH₃)CH(CH₃)CH(CH₃)(CH₂CH₃) and others. The phrase also includes cyclic alkyl groups such as cyclopropyl, cyclobutyl, cyclopentyl, cyclohexyl, cycloheptyl, and cyclooctyl and such rings substituted with straight and branched chain alkyl groups as defined above. The phrase also includes polycyclic alkyl groups such as, but not limited to, adamantyl norbornyl, and bicyclo[2.2.2]octyl and such rings substituted with straight and branched chain alkyl groups as defined above.

As used herein, the term “alkylene”, whether used alone or as part of a substituent group, includes any group obtained by removing a hydrogen atom from an alkyl group; an alkylene group forms two bonds with other groups.

As used herein, the term “alkenyl”, whether used alone or as part of a substituent group, includes an alkyl group having at least one double bond between any two adjacent carbon atoms.

As used herein, the term “unsubstituted alkyl” and “unsubstituted alkenyl” refers to alkyl and alkenyl groups that do not contain heteroatoms.

The phrase “substituted alkyl” and “substituted alkenyl” refers to alkyl and alkenyl groups as defined above in which one or more bonds to a carbon(s) or hydrogen(s) are replaced by a bond to non-hydrogen or non-carbon atoms such as, but not limited to, a halogen atom in halides such as F, Cl Br, and I; and oxygen atom in groups such as carbonyl, carboxyl, hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, enamines imines, oximes, hydrazones, and nitriles; a silicon atom in groups such as in trialkylsilyl groups, dialkylarylsilyl groups, alkyldiarylsilyl groups, and triarylsilyl groups; and other heteroatoms in various other groups. Other alkyl groups include those in which one or more bonds to a carbon or hydrogen atom is replaced by a bond to an oxygen atom such that the substituted alkyl group contains a hydroxyl, alkoxy, aryloxy group, or heterocyclyloxy group. Still other alkyl groups include alkyl groups that have an ainine, alkylamine, dialkylamine, arylamine, (alkyl)(aryl)amine, diarylamine, heterocyclylamine, (alkyl)(heterocyclyl)-amine, (aryl)(heterocyclyl)amine, or diheterocyclylamine group.

### Disaccharide Synthetic Lipid Compounds

The present disclosure provides synthetic disaccharide lipid compounds of the general structures I to V below. In one embodiment, the synthetic disaccharide lipid compounds contain 6 acyl groups at the positions disclosed herein. In another embodiment, the disclosed synthetic disaccharide lipid compounds are 3-O-deacyl disaccharide compounds (lacking an acyl group at the 3 position). In one embodiment, the 6 acyl chains are positioned at the 2′, 3′ and 2 positions of non-reducing and reducing sugars, respectively. In another embodiment, 2 acyl chains are present at each of the 2′, 3′ and 2 positions of non-reducing and reducing sugars, respectively. The length of the acyl chains present at each position may be varied as described herein. In certain embodiments, all of the acyl chains present at the 2′, 3′ and 2 positions of non-reducing and reducing sugars, respectively, have the same chain length (i.e., the same number of carbon atoms). In certain embodiments, the acyl chains present at the 2′, 3′ and 2 positions of non-reducing and reducing sugars, respectively, have different chain lengths (i.e., different numbers of carbon atoms). In certain embodiments, all but 1-3 of the acyl chains present at the 2′, 3′ and 2 positions of non-reducing and reducing sugars, respectively, have the same chain length (i.e., the same number of carbon atoms). In certain embodiments, all but 1-2 of the acyl chains present at the 2′, 3′ and 2 positions of non-reducing and reducing sugars, respectively, have the same chain length (i.e., the same number of carbon atoms). In certain embodiments, all but 1 of the acyl chains present at the 2′, 3′ and 2 positions of non-reducing and reducing sugars, respectively, have the same chain length (i.e., the same number of carbon atoms). In another embodiment of the foregoing, the compounds are mono-phosphoryl. In a further particular embodiment of the foregoing, the compounds are mono-phosphoryl with the phosphate group being present on the 4′ position. Such compounds are useful as immunostimulants for inducing and stimulating an immune response and are useful as adjuvants in immunogenic compositions such as, but not limited to, vaccines.

The compounds of the present disclosure are chemically synthesized and are therefore provided in essentially pure form. By “essentially pure”, it is meant that the synthetic disaccharide lipid compounds are at least 80%, at least 85%, at least 90%, at least 95%, at least 96%, at least 97%, at least 98% or at least 99% pure with respect to the synthetic disaccharide lipid compounds (measured on a weight basis). In a particular aspect, the synthetic disaccharide lipid compounds are at least 95% pure (measured on a weight basis). In another aspect, the synthetic disaccharide lipid compounds are at least 96% pure (measured on a weight basis). In another aspect, the synthetic disaccharide lipid compounds are at least 97% pure (measured on a weight basis). In another aspect, the synthetic disaccharide lipid compounds are at least 98% pure (measured on a weight basis). In another aspect, the synthetic disaccharide lipid compounds are at least 99% pure (measured on a weight basis).

This level of purity allows the synthetic disaccharide lipid compounds of the present disclosure to be used in various pharmacologic applications for which naturally purified lipid A compounds are unsuitable. For instance, naturally purified lipid A compounds are co-purified with various amounts of proteins, nucleic acids, other lipids and other products from the bacterial cells from which they are purified. In addition, the levels of such impurities vary from purification to purification. Furthermore, the naturally purified lipid A compounds are often present in various forms. For instance, the number of acyl chains present on the disaccharide backbone may vary in a given preparation, as well as the length of a given acyl chain at a particular position. Therefore, the compounds of the present disclosure are essentially free of contaminants found in compounds purified from natural sources, such as, but not limited to, proteins, nucleic acids, other lipids and other products from a bacterial cell. In addition, the compounds of the present disclosure are essentially free of contaminants generated during chemical synthesis. As such, the compounds of the present disclosure provide an advantage over the compounds known in the art.

As used herein, the term “essentially free” means that the compounds of the present disclosure contain less than 1%, less than 2%, less than 3%, less than 4%, less than 5%, less than 10%, less than 15% or less than 20% of contaminants as determined with respect to the synthetic disaccharide lipid compound (as measured on a weight basis). Relevant contaminants include, but are not limited to, related compounds having different number and/or length of the acyl chains, those contaminants encountered during isolation of corresponding compounds from natural sources and contaminants encountered as a result of chemical synthesis. In one aspect, the synthetic disaccharide lipid compounds contain less than 1% of such contaminants as determined with respect to the synthetic disaccharide lipid compound (as measured on a weight basis). In one aspect, the synthetic disaccharide lipid compounds contain less than 2% of such contaminants as determined with respect to the synthetic disaccharide lipid compound (as measured on a weight basis). In one aspect, the synthetic disaccharide lipid compounds contain less than 3% of such contaminants as determined with respect to the synthetic disaccharide lipid compound (as measured on a weight basis). In one aspect, the synthetic disaccharide lipid compounds contain less than 4% of such contaminants as determined with respect to the synthetic disaccharide lipid compound (as measured on a weight basis). In one aspect, the synthetic disaccharide lipid compounds contain less than 5% of such contaminants as determined with respect to the synthetic disaccharide lipid compound (as measured on a weight basis).

The foregoing concerns result in the properties of the naturally purified lipid A compounds being variable from preparation to preparation. Furthermore, individual subject reaction to such naturally occurring lipid A compounds may vary as well. The provision of the compounds of the present disclosure in an essentially pure form reduces the foregoing concerns and allows the use of compounds of the present disclosure in applications in which naturally occurring lipid A compositions are unsuited.

The art is also aware of various mono-phosphorylated hexaacyl disaccharide compounds. For example, such compounds are provided under the name PHAD (or PHAD™) and are available from Avanti Polar Lipids (Alabaster, Ala.; catalog number 699800). However, these compounds have an acyl group at the 3 position.

3-O-deacyl mono-phosphorylated disaccharide compounds, including, but not limited to, pentaacyl compounds, are known in the art. However, such 3-O-deacyl mono-phosphorylated disaccharide compounds have been purified from natural sources and treated chemically to remove the acyl chain present at the 3 position. As a result, the variability in the composition of the prior art 3-O-deacyl mono-phosphorylated disaccharide compounds is relevant. In addition, the foregoing, chemical modification procedure also introduces additional variability.

Therefore, the prior art has not provided synthetic disaccharide lipid compounds in an essentially pure form and/or essentially free from contaminants A representative disaccharide compound with the 2′, 3′, 2 and 3 positions labeled is shown below.

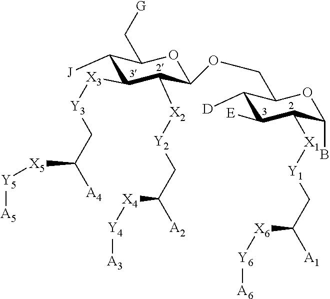

For example, an exemplary synthetic disaccharide lipid compounds of the present disclosure may contain 2 acyl chains at the 3′ position, 2 acyl chains at the 2 position and 2 acyl chains at the 2′ position (for a total of 6 acyl chains).

In one embodiment, the length of the acyl chains may vary from 6 to 19 carbons in length. For clarity, the length of the 6 acyl chains present in the disclosed synthetic disaccharide lipid compounds may each be the same or may be different. In a particular embodiment, the length of the 6 acyl chains is the same. In another particular embodiment, the length of all but 2 of the 6 acyl chains is the same (the length of the 2 acyl chains may be the same or may be different from one another). In another particular embodiment, the length of all but 1 of the 6 acyl chains is the same. The 6 acyl chains may be saturated and contain no double bonds or the 6 acyl chains may be unsaturated. When such acyl chains are unsaturated, each unsaturated acyl chain may contain from 1 to 3 double bonds. In one embodiment, the 6 acyl chains are all saturated. In another embodiment, at least one of the 6 acyl chains is unsaturated and contains a single double bond and the remainder of the acyl chains are saturated.

In one embodiment, such synthetic disaccharide lipid compounds have the general structure shown in formula I:

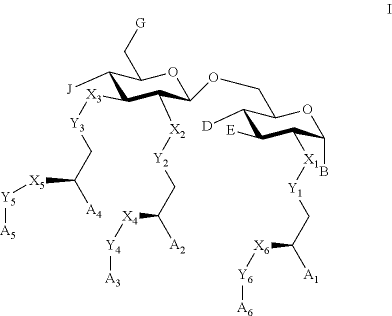

or a pharmaceutically acceptable salt or prodrug thereof, wherein:
X₁, X₂, X₃, X₄, X₅ and X₆ are each independently absent or selected from —O—, —NH— or —CH₂—;
Y₁, Y₂, Y₃, Y₄, Y₅ and Y₆ are each independently selected from H, —CH₂—, or —C(═O)—, provided that at least 3 of Y₁, Y₂, Y₃, Y₄, Y₅ and Y₆ are a group other than H and further provided that when one of Y₁, Y₂, Y₃, Y₄, Y₅ and Y₆ is H, then the groups which are linked, directly or indirectly, to Y₁, Y₂, Y₃, Y₄, Y₅ and Y₆ are absent;
D, E and G are each independently selected from C₁-C₄ alkyl, —OH, —SH, —OC(═O)(CH₂)ₘ—CH₃, —OC(═O)(CH₂)ₙC(═O)OH, —OC(═O)CH(NH₂)(CH₂)ₙC(═O)OH or
—OC(═O)CH₂CH(OH)(CH₂)ₙCH₃;
J and B are each independently selected from OH, OR₁, H, —OP(═O)(OH)₂, —OP(═O)(OH)(OR₂), —OP(═O)(OR₂)₂, —OS(═O)(OH)₂, —OS(═O)(OR₂)₂, —OS(OH)₂, —OS(OR₂)₂, —C(═O)OH, —C(═O)OR₂ or an acidic group;
A₁, A₂, and A₄ are each independently selected from C₆ to C₂₀ substituted or unsubstituted alkyl or alkenyl;
A₃, A₅ and A₆ are each independently selected from C₈ to C₂₂ substituted or unsubstituted alkyl or alkenyl;
R₁ is a substituted or unsubstituted C₁-C₄ alkyl;
R₂ is independently for each occurrence, H, OH, alkyl, substituted alkyl or N-linked amino acid residue; and
m and n are each independently an integer from 0 to 5.

In first aspect of this embodiment, X₁, X₂ and X₃ are selected from —O— or —NH—. Still further, all of X₁, X₂ and X₃ are —O— or all of X₁, X₂ and X₃ are —NH—. Still further one of X₁, X₂ and X₃ is —O— and the remaining two of X₁, X₂ and X₃ are —NH—.

In a second aspect of this embodiment, at least one of X₃, X₄, X₅ and X₆ are —O—, at least two of X₃, X₄, X₅ and X₆ are —O—, at least three of X₃, X₄, X₅ and X₆ are —O— or all of X₃, X₄, X₅ and X₆ are —O—.

In a third aspect of this embodiment, at least one of X₁ and X₂ are —NH—, or both of X₁ and X₂ are —NH—.

In a fourth aspect of this embodiment, X₃, X₄, X₅ and X₆ are —O— and X₁ and X₂ are —NH—.

In a fifth aspect of this embodiment, X₁ to X₅ are as defined in the first through fourth aspects and at least one of Y₁, Y₂, Y₃, Y₄, Y₅ and Y₆ are —C(═O)—, at least two of Y₁, Y₂, Y₃, Y₄, Y₅ and Y₆ are —C(═O)—, at least three of Y₁, Y₂, Y₃, Y₄, Y₅ and Y₆ are —C(═O)—, at least four of Y₁, Y₂, Y₃, Y₄, Y₅ and Y₆ are —C(═O)—, at least five of Y₁, Y₂, Y₃, Y₄, Y₅ and Y₆ are —C(═O)—, or all of Y₁, Y₂, Y₃, Y₄, Y₅ and Y₆ are —C(═O)—.

In a sixth aspect of this embodiment, X₃, X₄, X₅ and X₆ are —O— and X₁ and X₂ are —NH— and all of Y₁, Y₂, Y₃, Y₄ and Y₅ are —C(═O)—.

In a seventh aspect of this embodiment, X₁ to X₆ are as defined in the first through sixth aspects, Y₁ to Y₆ are as defined in the fifth and sixth aspects and J is —OP(═O)(OH)₂ and B is —OH.

In an eight aspect of this embodiment, X₁ to X₆ are as defined in the first through sixth aspects, Y₁ to Y₆ are as defined in the fifth and sixth aspects, J and B are defined as in the seventh aspect and at least one of D, E and G is OH, at least two of D, E and G are OH or D, E and G are each OH.

In a ninth aspect of this embodiment, X₁ to X₆ are as defined in the first through sixth aspects, Y₁ to Y₆ are as defined in the fifth and sixth aspects, J and B are defined as in the seventh aspect, D, E and G are defined as in the eighth aspect and A₁ A₂, and A₄ are each independently C₉ to C₁₅ unsubstituted alkyl and A₃, A₅ and A₆ are each independently are C₁₁ to C₁₇ unsubstituted alkyl.

In any of the first through eighth aspects of this embodiment, A₁ A₂, and A₄ are each C₁₁ unsubstituted alkyl and A₃, A₅ and A₆ are each C₁₃ unsubstituted alkyl.

In any of the first through eighth aspects of this embodiment, A₁ A₂, and A₄ are each C₁₁ unsubstituted alkyl, A₃ is a C₁₁ unsubstituted alkyl and A₅ and A₆ are each C₁₃ unsubstituted alkyl.

In any of the first through eighth aspects of this embodiment, A₁ A₂, and A₄ are each C₁₁ unsubstituted alkyl, A₆ is a C₁₅ unsubstituted alkyl and A₅ and A₃ are each C₁₃ unsubstituted alkyl.

In any of the first through eighth aspects of this embodiment, A₁ A₂, and A₄ are each C₁₁ unsubstituted alkyl, A₆ is a C₁₅ unsubstituted alkyl and A₃ is a C₁₁ unsubstituted alkyl and A₅ is a C₁₃ unsubstituted alkyl.

In another embodiment, such synthetic disaccharide lipid compounds have the general structure shown in formula II:

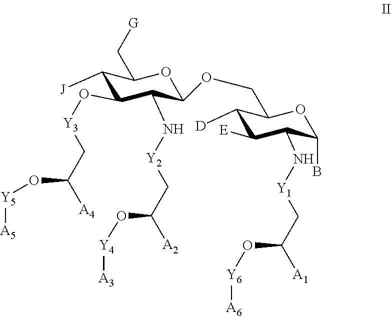

or a pharmaceutically acceptable salt or prodrug thereof, wherein:
Y₁, Y₂, Y₃, Y₄, Y₅ and Y₆ are each independently selected from H, —CH₂—, or —C(═O)—, provided that at least 3 of Y₁, Y₂, Y₃, Y₄, Y₅ and Y₆ are a group other than H and further provided that when one of Y₁, Y₂, Y₃, Y₄, Y₅ and Y₆ is H, then the groups which are linked, directly or indirectly, to Y₁, Y₂, Y₃, Y₄, Y₅ and Y₆ are absent;
D, E and G are each independently selected from C₁-C₄ alkyl, —OH, —SH, —OC(═O)(CH₂)ₘ—CH₃, —OC(═O)(CH₂)ₙC(═O)OH, —OC(═O)CH(NH₂)(CH₂)ₙC(═O)OH or
—OC(═O)CH₂CH(OH)(CH₂)ₙCH₃;
J and B are each independently selected from OH, OR₁, H, —OP(═O)(OH)₂, —OP(═O)(OH)(OR₂), —OP(═O)(OR₂)₂, —OS(═O)(OH)₂, —OS(═O)(OR₂)₂, —OS(OH)₂, —OS(OR₂)₂, —C(═O)OH, —C(═O)OR₂ or an acidic group;
A₁, A₂, and A₄ are each independently selected from C₆ to C₂₀ substituted or unsubstituted alkyl or alkenyl;
A₃, A₅ and A₆ are each independently selected from C₈ to C₂₂ substituted or unsubstituted alkyl or alkenyl;
R₁ is a substituted or unsubstituted C₁-C₄ alkyl;
R₂ is independently for each occurrence, H, OH, alkyl, substituted alkyl or N-linked amino acid residue; and
m and n are each independently an integer from 0 to 5.

In a first aspect of this embodiment, at least one of Y₁, Y₂, Y₃, Y₄, Y₅ and Y₆ are —C(═O), at least two of Y₁, Y₂, Y₃, Y₄, Y₅ and Y₆ are —C(═O)—, at least three of Y₁, Y₂, Y₃, Y₄, Y₅ and Y₆ are —C(═O)—, at least four of Y₁, Y₂, Y₃, Y₄, Y₅ and Y₆ are —C(═O)—, at least five of Y₁, Y₂, Y₃, Y₄, Y₅ and Y₆ are —C(═O)—, or all of Y₁, Y₂, Y₃, Y₄, Y₅ and Y₆ are —C(═O)—.

In a second aspect of this embodiment, Y₁ to Y₆ are as defined in the first aspect and J is —OP(═O)(OH)₂ and B is —OH.

In third aspect of this embodiment, Y₁ to Y₆ are as defined in the first aspect, J and B are defined as in the second aspect and at least one of D, E and G is OH, at least two of D, E and G are OH or D, E and G are each OH.

In a fourth aspect of this embodiment, Y₁ to Y₆ are as defined in the first aspect, J and B are defined as in the second aspect, D, E and G are defined as in the third aspect and A₁ A₂, and A₄ are each independently C₉ to C₁₅ unsubstituted alkyl and A₃, A₅ and A₆ are each independently are C₁₁ to C₁₇ unsubstituted alkyl.

In any of the first through fourth aspects of this embodiment, A₁ A₂, and A₄ are each C₁₁ unsubstituted alkyl and A₃, A₅ and A₆ are each C₁₃ unsubstituted alkyl.

In any of the first through fourth aspects of this embodiment, A₁, A₂, and A₄ are each C₁₁ unsubstituted alkyl, A₃ is a C₁₁ unsubstituted alkyl and A₅ and A₆ are each C₁₃ unsubstituted alkyl.

In any of the first through fourth aspects of this embodiment, A₁, A₂, and A₄ are each C₁₁ unsubstituted alkyl, A₆ is a C₁₅ unsubstituted alkyl and A₅ and A₃ are each C₁₃ unsubstituted alkyl.

In any of the first through fourth aspects of this embodiment, A₁ A₂, and A₄ are each C₁₁ unsubstituted alkyl, A₆ is a C₁₅ unsubstituted alkyl and A₃ is a C₁₁ unsubstituted alkyl and A₅ is a C₁₃ unsubstituted alkyl.

In still another embodiment, synthetic disaccharide lipid compounds have the general structure shown in formula III:

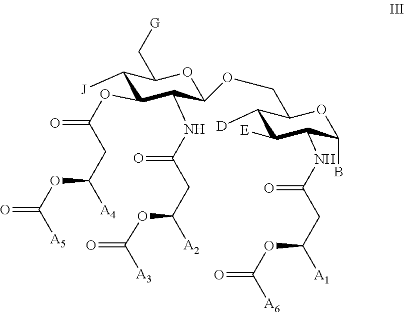

or a pharmaceutically acceptable salt or prodrug thereof, wherein:
D, E and G are each independently selected from C₁-C₄ alkyl, —OH, —SH, —OC(═O)(CH₂)ₘ—CH₃, —OC(═O)(CH₂)ₙC(═O)OH, —OC(═O)CH(NH₂)(CH₂)ₙC(═O)OH or
—OC(═O)CH₂CH(OH)(CH₂)ₙCH₃;
J and B are each independently selected from OH, OR₁, H, —OP(═O)(OH)₂, —OP(═O)(OH)(OR₂), —OP(═O)(OR₂)₂, —OS(═O)(OH)₂, —OS(═O)(OR₂)₂, —OS(OH)₂, —OS(OR₂)₂, —C(═O)OH, —C(═O)OR₂ or an acidic group;
A₁, A₂, and A₄ are each independently selected from C₆ to C₂₀ substituted or unsubstituted alkyl or alkenyl;
A₃. A₅ and A₆ are each independently selected from C₈ to C₂₂ substituted or unsubstituted alkyl or alkenyl;
R₁ is a substituted or unsubstituted C₁-C₄ alkyl;
R₂ is independently for each occurrence, H, OH, alkyl, substituted alkyl or N-linked amino acid residue; and
m and n are each independently an integer from 0 to 5.

In a first aspect of this embodiment, J is —OP(═O)(OH)₂ and B is —OH.

In a second aspect of this embodiment, J and B are defined as in the first aspect and at least one of D, E and G is OH, at least two of D, E and G are OH or D, E and G are each OH.

In a third aspect of this embodiment, J and B are defined as in the first aspect, D, E and G are defined as in the second aspect and A₁ A₂, and A₄ are each independently C₉ to C₁₅ unsubstituted alkyl and A₃, A₅ and A₆ are each independently are C₁₁ to C₁₇ unsubstituted alkyl.

In any of the first through third aspects of this embodiment, A₁ A₂, and A₄ are each C₁₁ unsubstituted alkyl and A₃, A₅ and A₆ are each C₁₃ unsubstituted alkyl.

In any of the first through third aspects of this embodiment, A₁, A₂, and A₄ are each C₁₁ unsubstituted alkyl, A₃ is a C₁₁ unsubstituted alkyl and A₅ and A₆ are each C₁₃ unsubstituted alkyl.

In any of the first through third aspects of this embodiment, A₁ A₂, and A₄ are each C₁₁ unsubstituted alkyl, A₆ is a C₁₅ unsubstituted alkyl and A₅ and A₃ are each C₁₃ unsubstituted alkyl.

In any of the first through third aspects of this embodiment, A₁ A₂, and A₄ are each C₁₁ unsubstituted alkyl, A₆ is a C₁₅ unsubstituted alkyl and A₃ is a CH unsubstituted alkyl and A₅ is a C₁₃ unsubstituted alkyl.

In still another embodiment, such synthetic disaccharide lipid compounds have the general structure shown in formula IV:

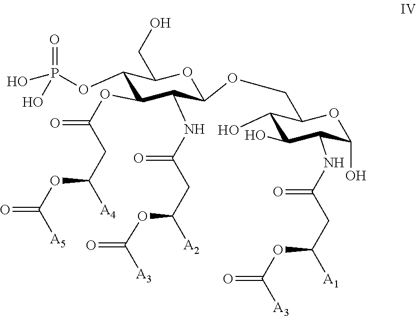

or a pharmaceutically acceptable salt or prodrug thereof, wherein:
A₁, A₂, and A₄ are each independently selected from C₆ to C₂₀ substituted or unsubstituted alkyl or alkenyl; and
A₃, A₅ and A₆ are each independently selected from C₈ to C₂₂ substituted or unsubstituted alkyl or alkenyl;

In a first aspect of this embodiment, A₁ A₂, and A₄ are each independently C₉ to C₁₅ unsubstituted alkyl and A₃, A₅ and A₆ are each independently are C₁₁ to C₁₇ unsubstituted alkyl.

In a second aspect of this embodiment, A₁ A₂, and A₄ are each C₁₁ unsubstituted alkyl and A₃, A₅ and A₆ are each C₁₃ unsubstituted alkyl.

In a third aspect of this embodiment, A₁, A₂, and A₄ are each C₁₁ unsubstituted alkyl, A₃ is a C₁₁ unsubstituted alkyl and A₅ and A₆ are each C₁₃ unsubstituted alkyl.

In a fourth of this embodiment, A₁ A₂, and A₄ are each C₁₁ unsubstituted alkyl, A₆ is a C₁₅ unsubstituted alkyl and A₅ and A₃ are each C₁₃ unsubstituted alkyl.

In a fifth of this embodiment, A₁ A₂, and A₄ are each C₁₁ unsubstituted alkyl, A₆ is a C₁₅ unsubstituted alkyl and A₃ is a C₁₁ unsubstituted alkyl and A₅ is a C₁₃ unsubstituted alkyl.

In yet another embodiment, such synthetic disaccharide lipid compound has the structure shown in formula Va, Vb, Vc and Vd, or a pharmaceutically acceptable salt or prodrug thereof:

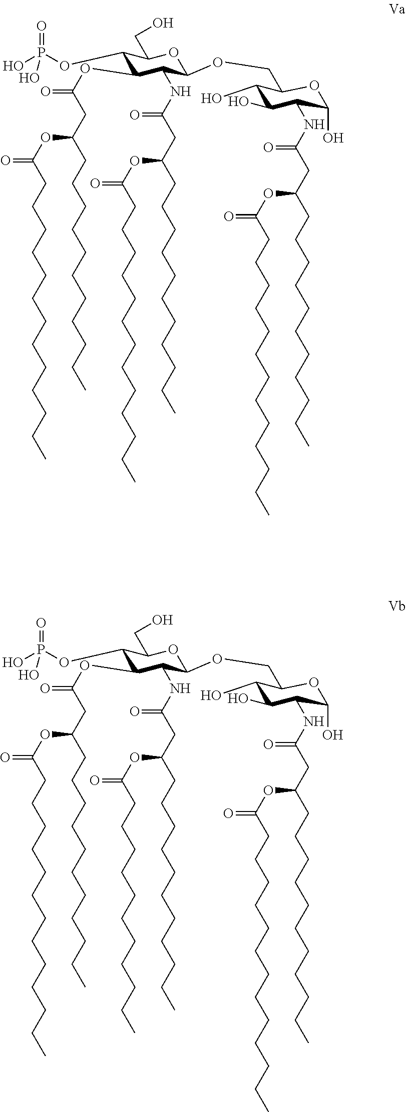

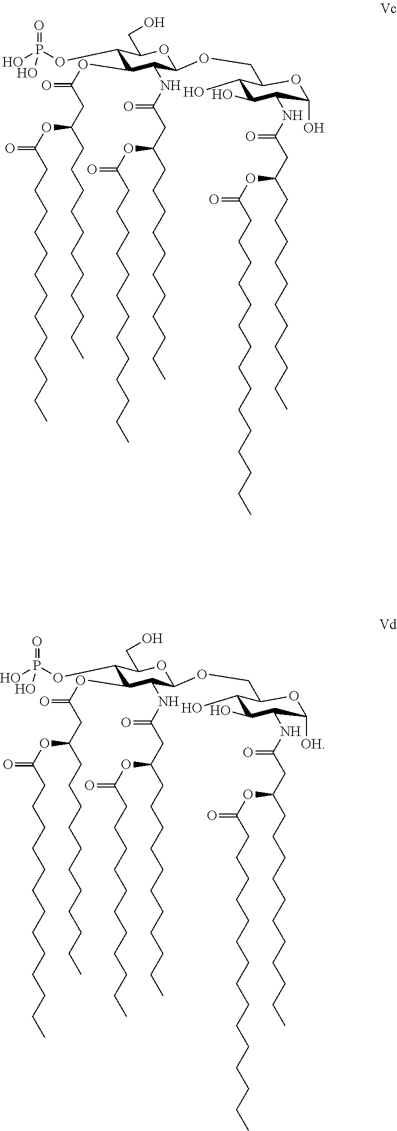

### Methods of Using the Compounds of the Present Disclosure

The present disclosure shows that disaccharide synthetic lipid compounds may be prepared in an essentially pure form. Therefore, the provision of an essentially pure synthetic disaccharide lipid compounds of the present disclosure allows for methods of stimulating an immune response in a subject that are free from the disadvantages of the compounds known in the art that may contain impurities or be mixtures of compounds (for example compounds with a mixture of acylation patters or a mixture of different acyl chain lengths at a given position).

In one embodiment, the present disclosure provides methods for stimulating or eliciting an immune response in a subject. Such method comprises the step of administering to the subject an amount of a synthetic disaccharide lipid compound of the present disclosure or a pharmacologically acceptable salt thereof. In one aspect, the synthetic disaccharide lipid compound or a pharmacologically acceptable salt thereof is administered alone. In another aspect, the synthetic disaccharide lipid compound or a pharmacologically acceptable salt thereof is administered with a second adjuvant or additional adjuvants. In another aspect, the synthetic disaccharide lipid compound or a pharmacologically acceptable salt thereof is administered with an antigen. In another aspect, the synthetic disaccharide lipid compound or a pharmacologically acceptable salt thereof is administered with an antigen and a second adjuvant or additional adjuvants. In one embodiment, such administration increases an immune response in a subject. When an antigen is included, such administration increases an immune response in a subject that is specific, at least in part, to the antigen delivered.

In one embodiment, the present disclosure provides methods for enhancing an immune response in a subject. Such method comprises the step of administering to the subject an amount of a synthetic disaccharide lipid compound of the present disclosure or a pharmacologically acceptable salt thereof. In one aspect, a synthetic disaccharide lipid compound or a pharmacologically acceptable salt thereof is administered alone. In another aspect, the synthetic disaccharide lipid compound or a pharmacologically acceptable salt thereof is administered with a second adjuvant or additional adjuvants. In another aspect, the synthetic disaccharide lipid compound or a pharmacologically acceptable salt thereof is administered with an antigen. In another aspect, the synthetic disaccharide lipid compound or a pharmacologically acceptable salt thereof is administered with an antigen and a second adjuvant or additional adjuvants. In one embodiment, such administration enhances an immune response in a subject. When an antigen is included, such administration enhances an immune response in a subject that is specific, at least in part, to the antigen delivered.

In one embodiment, the present disclosure provides methods for stimulating immunoglobulin production in a subject. In one embodiment, the immunoglobulin is IgG. In another embodiment, the immunoglobulin is IgM. Such method comprises the step of administering to the subject an amount of a synthetic disaccharide lipid compound of the present disclosure or a pharmacologically acceptable salt thereof. In one aspect, a synthetic disaccharide lipid compound or a pharmacologically acceptable salt thereof is administered alone. In another aspect, the synthetic disaccharide lipid compound or a pharmacologically acceptable salt thereof is administered with a second adjuvant or additional adjuvants. In another aspect, the synthetic disaccharide lipid compound or a pharmacologically acceptable salt thereof is administered with an antigen. In another aspect, the synthetic disaccharide lipid compound or a pharmacologically acceptable salt thereof is administered with an antigen and a second adjuvant or additional adjuvants. In one embodiment, such administration stimulates or enhances an immune response in a subject. When an antigen is included, the immunoglobulin produced may be specific to the antigen delivered.

Toll-Like Receptors (TLRs), including TLR4, are pattern-recognition receptors (PRRs). TLRs play a well-known role in the initiation of immune responses. At least 10 functional TLRs have been identified in humans. Each TLR detects distinct pathogen associated molecular patterns derived from viruses, bacteria, mycobacteria, fungi, and parasites. Gram-negative bacteria are typically sensed through the cell wall constituent lipopolysaccharide (LPS) that binds in complex with the LPS-binding Protein (LBP) to a receptor complex of TLR4, CD14 and an associated protein (MD-2). The TLR4-mediated signalling cascades then modulate the gene expression towards the production of a variety of pro-inflammatory cytokines such as Interleukin (IL)-6, Tumour necrosis factor (TNF)-α and IL-12. In addition, these signalling events enhance the co-stimulatory function of monocytes.

In one embodiment, the present disclosure provides methods for stimulating TLR4 and/or stimulating a TLR4 response. Stimulating a TLR4 response includes stimulating TLR4 signaling Such method comprises the step of administering to the subject an amount of a synthetic disaccharide lipid compound of the present disclosure or a pharmacologically acceptable salt thereof. In one aspect, the synthetic disaccharide lipid compound or a pharmacologically acceptable salt thereof is administered alone. In another aspect, the synthetic disaccharide lipid compound or a pharmacologically acceptable salt thereof is administered with a second adjuvant or additional adjuvants. In another aspect, the synthetic disaccharide lipid compound or a pharmacologically acceptable salt thereof is administered with an antigen. In another aspect, the synthetic disaccharide lipid compound or a pharmacologically acceptable salt thereof is administered with an antigen and a second adjuvant or additional adjuvants. In one embodiment, such administration stimulates or enhances an immune response in a subject. When an antigen is included, such administration stimulates or enhances an immune response in a subject that is specific, at least in part, to the antigen delivered.

In one embodiment, the present disclosure provides for a pharmaceutical composition, such as a vaccine, comprising a synthetic disaccharide lipid compound of the present disclosure in combination with an antigen and an optional second adjuvant and other components as described herein. In such an aspect, the pharmaceutical composition stimulates a specific immune response in a subject for the purpose of treating and/or preventing a disease or condition in a subject.

In one aspect of the foregoing embodiments, the present disclosure provides for monotherapy using a synthetic disaccharide lipid compound of the present disclosure alone (i.e., without the addition of an antigen or other immune response modifiers). In such an aspect, the synthetic disaccharide lipid compound stimulates a non-specific immune response in a subject for the purpose of treating and/or preventing a disease or condition in a subject.

In one aspect of the foregoing embodiments, the present disclosure provides for therapy using a synthetic disaccharide lipid compound of the present disclosure in combination with a second adjuvant (but without the addition of an antigen). In such an aspect, the synthetic disaccharide lipid compound and second adjuvant stimulates a non-specific immune response in a subject for the purpose of treating and/or preventing a disease or condition in a subject.

In one aspect of the foregoing embodiments, the synthetic disaccharide lipid compound is a compound of the general formula (I). In another aspect of these embodiments, the synthetic disaccharide lipid compound is a compound of the general formula (II). In another aspect of these embodiments, the synthetic disaccharide lipid compound is a compound of the general formula (III). In another aspect of these embodiments, the synthetic disaccharide lipid compound is a compound of the general formula (IV). In another aspect of these embodiments, the synthetic disaccharide lipid compound is a compound of the general formula (Va). In another aspect of these embodiments, the synthetic disaccharide lipid compound is a compound of the general formula (Vb). In another aspect of these embodiments, the synthetic disaccharide lipid compound is a compound of the general formula (Vc). In another aspect of these embodiments, the synthetic disaccharide lipid compound is a compound of the general formula (Vd).

In one aspect of the foregoing embodiments, the synthetic disaccharide lipid compound is a compound of the general formula (Va). In one aspect of these embodiments, the synthetic disaccharide lipid compound is a compound of the general formula (Vc).

In one aspect of the foregoing embodiments, the second adjuvant is any compound that has an immunostimulatory effect that is not a synthetic disaccharide lipid compound of the present disclosure. As used herein, the term immunostimulatory and similar terms means that a compound or composition provides agent enhances a subject's immune response, either in a general manner or in response to an antigen.

In one aspect of the foregoing embodiment, the second adjuvant is a TLR agonist.

In one aspect of the foregoing embodiments, the second adjuvant is Monophosphoryl Lipid A (also known as mono-phosphorylated hexaacyl disaccharide and PHAD™) (Avanti Polar Lipids, Alabaster Ala.; catalogue number 699800).

In one aspect of the foregoing embodiments, the second adjuvant is Monophosphoryl 3-Deacyl Lipid A (also known as 3-D PHAD™) (Avanti Polar Lipids, Alabaster Ala.; catalogue number 699852).

In one aspect of the foregoing embodiments, the second adjuvant is a compound as described in US Patent Publication 2015-0110854-A1, which is hereby incorporated by reference for such teachings.

In one aspect of the foregoing embodiments, the synthetic disaccharide lipid compound may be administered alone or as a part of a pharmaceutical composition as described herein. A single compound of the general formula (I) to (V) may be administered; multiple compounds of the general formula (I) to (V) may be administered.

In one aspect of the foregoing embodiments, the subject is determined to be in need of such treatment. In a further aspect of these embodiments, the synthetic disaccharide lipid compound is administered in a therapeutically effective amount. Furthermore, in one aspect of the methods described above, the synthetic disaccharide lipid compound is a compound of the general formula Va-Vd. In one aspect of the methods described above, the synthetic disaccharide lipid compound is a compound of the general formula Va. In one aspect of the methods described above, the synthetic disaccharide lipid compound is a compound of the general formula Vc.

In the methods disclosed herein, the subject may be a mammal. In certain embodiments, the subject is a human.

The compounds and pharmaceutical compositions can be administered in a variety of dosage ranges. In one aspect of the foregoing embodiments, the dosage of the synthetic disaccharide lipid compound is from about 0.0001 μg/kg to about 5 mg/kg. In another aspect of the foregoing embodiments, the dosage of the disaccharide synthetic lipid compound is from about 0.01 μg/kg to about 2 mg/kg. In another aspect of the foregoing embodiments, the dosage of the disaccharide synthetic lipid compound is from about 0.1 μg/kg to about 1 mg/kg. In another aspect of the foregoing embodiment, the dosage of the disaccharide synthetic lipid compound is from about 0.1 μg/kg to about 0.1 mg/kg. In another aspect of the foregoing embodiment, the dosage of the synthetic disaccharide lipid compound is from about 1 ug/kg to about 50 μg/kg. In another aspect of the foregoing embodiment, the dosage of the synthetic disaccharide lipid compound is from about 1 μg/kg to about 25 μg/kg. In another aspect of the foregoing embodiment, the dosage of the synthetic disaccharide lipid compound is from about 1 ug/kg to about 15 ug/kg. In another aspect of the foregoing embodiment, the dosage of the synthetic disaccharide lipid compound is from about 0.001 μg/kg to about 15 μg/kg. In another aspect of the foregoing embodiment, the dosage of the synthetic disaccharide lipid compound is from about 0.01 μg/kg to about 15 μg/kg. In another aspect of the foregoing embodiment the dosage of the synthetic disaccharide lipid compound is from about 0.1 μg/kg to about 15 μg/kg.

In the methods described herein, the subjects treated can be further treated with one or more additional active agents. These additional active agents may be delivered together with or separate from the synthetic disaccharide lipid compounds of the present disclosure. It will be evident to those skilled in the art that the number and frequency of administration will be dependent upon the response of the subject.

### Antigen

An antigen, for use in certain embodiments described herein, may be any molecule or molecule complex that generates an immune response. In one embodiment, the molecule or molecule complex generates a weak or incomplete immune response. In one embodiment, the antigen is a target epitope to which an immune response is desired, a molecule (including a biomolecule such as a polypeptide or nucleic acid), a molecular complex (including molecular complexes that contain biomolecules), a sub-cellular fraction, cell or tissue (or a fraction of either) against which elicitation of an immune response is desired. When a polypeptide is an antigen, the polypeptide may be naturally occurring or recombinant. In one embodiment, the vaccine formulations of the present invention contains an antigen or antigenic composition capable of eliciting an immune response against a human or mammalian pathogen; in such an embodiment the antigen may be derived from such pathogen or be an antigen which cross reacts with such pathogen.

### Pharmaceutical Compositions

The present disclosure provides various pharmaceutical compositions. In one embodiment, the pharmaceutical compositions of the present disclosure comprise, consist of or consist essentially of at least one synthetic disaccharide lipid compound of the present disclosure and a pharmaceutically acceptable carrier, excipient or diluent. The pharmaceutical compositions of the present disclosure may further comprise additional agents, such as, but not limited to, second adjuvants and an antigen (including a plurality of antigens). When a plurality of antigens are present, each antigen may be directed to different epitopes or the same epitope. In certain embodiments, the pharmaceutical composition lacks an antigen. In certain embodiments, the pharmaceutical composition contains at least one adjuvant.

In one embodiment, the pharmaceutical compositions of the present disclosure comprise, consist of or consist essentially of at least one synthetic disaccharide lipid compound of the present disclosure and a pharmaceutically acceptable carrier, excipient or diluent and lacks an antigen.

In one embodiment, the pharmaceutical compositions of the present disclosure comprise, consist of or consist essentially of at least one synthetic disaccharide lipid compound of the present disclosure, a pharmaceutically acceptable carrier, excipient or diluent and an antigen.

In one embodiment, the pharmaceutical compositions of the present disclosure comprise, consist of or consist essentially of at least one synthetic disaccharide lipid compound of the present disclosure, a pharmaceutically acceptable carrier, excipient or diluent and at least two antigens.

In another embodiment, the pharmaceutical compositions of the present disclosure comprise, consist of or consist essentially of at least one synthetic disaccharide lipid compound of the present disclosure and a pharmaceutically acceptable carrier, excipient or diluent and a second adjuvant and lacks an antigen.

In still another embodiment, the pharmaceutical compositions of the present disclosure comprise, consist of or consist essentially of at least one synthetic disaccharide synthetic lipid compound of the present disclosure, a pharmaceutically acceptable carrier, excipient or diluent, an antigen and a second adjuvant.

In still another embodiment, the pharmaceutical compositions of the present disclosure comprise, consist of or consist essentially of at least one synthetic disaccharide synthetic lipid compound of the present disclosure, a pharmaceutically acceptable carrier, excipient or diluent, at least two antigens and a second adjuvant.

In one aspect of the foregoing embodiments, the at least one synthetic disaccharide lipid compound of the present disclosure is a compound of the general structure (I). In another aspect of the foregoing embodiments, the at least one synthetic disaccharide lipid compound of the present disclosure is a compound of the general structure (II). In still another aspect of the foregoing embodiments, the at least one synthetic disaccharide lipid compound of the present disclosure is a compound of the general structure (III). In yet another aspect of the foregoing embodiments, the at least one synthetic disaccharide lipid compound of the present disclosure is a compound of the general structure (IV). In a further aspect of the foregoing embodiments, the at least one synthetic disaccharide lipid compound of the present disclosure is a compound of the general structure (Va). In one aspect of the foregoing embodiments, the at least one synthetic disaccharide lipid compound of the present disclosure is a compound of the general structure (Vb). In another aspect of the foregoing embodiments, the at least one synthetic disaccharide lipid compound of the present disclosure is a compound of the general structure (Vc). In still another aspect of the foregoing embodiments, the at least one synthetic disaccharide lipid compound of the present disclosure is a compound of the general structure (Vd).

In yet a further aspect of the foregoing embodiments, the at least one synthetic disaccharide lipid compound of the present disclosure is a compound of the general structure (Va). In yet a further aspect of the foregoing embodiments, the at least one synthetic disaccharide lipid compound of the present disclosure is a compound of the general structure (Vc).

In one aspect of the foregoing embodiments, the pharmaceutical composition is a vaccine composition. As set forth above, such a vaccine composition may contain only a synthetic disaccharide lipid compound of the present disclosure and a pharmaceutically acceptable carrier, excipient or diluent. As such, the immune stimulating effects will be provided by the synthetic disaccharide lipid compound. Furthermore, as set forth above, such a vaccine composition may contain only a synthetic disaccharide lipid compound of the present disclosure, a pharmaceutically acceptable carrier, excipient or diluent and an antigen. As such, the immune stimulating effects will be provided by the synthetic disaccharide lipid compound and/or the antigen. Furthermore, as set forth above, such a vaccine composition may contain only a synthetic disaccharide lipid compound of the present disclosure, a pharmaceutically acceptable carrier, excipient or diluent and a second adjuvant. As such, the immune stimulating effects will be provided by the synthetic disaccharide lipid compound and/or the second adjuvant. Furthermore, as set forth above, such a vaccine composition may contain only a synthetic disaccharide lipid compound of the present disclosure, a pharmaceutically acceptable carrier, excipient or diluent, an antigen and a second adjuvant. As such, the immune stimulating effects will be provided by the synthetic disaccharide lipid compound, the antigen and/or the second adjuvant. In any of the foregoing, when an antigen is present, more than one antigen may be present.

In one aspect of the foregoing embodiments, the second adjuvant is any adjuvant known in the art. The second adjuvant is a compound or compounds that display adjuvant activity when administered to a subject (i.e., altering, increasing or decreasing, the potency and/or longevity of an immune response as described in Powell and Newman, “Vaccine design—The Subunit and Adjuvant Approach”, 1995, Plenum Press, New York). Second adjuvants include, but are not limited to, saponins and saponin mimetics (such as but not limited to QS21, QS17, QS7 and mimetics), alum, plant alkaloids (such as but not limited to tomatine), detergents (such as but not limited to saponin, escin, digitonin polysorbate 80, Span 85 and stearyl tyrosine), block copolymer or biodegradable polymer (such as but not limited to Pluronic, L121, CRL1005, poly(lactic-co-glycolic acid), poly(lactic acid), poly-(D,L-lactide-co-glycolide) and polyinosinic:polycytidylic acid), one or more cytokines (such as but not limited to GM-CSF, IL-2, IL-7, IL-12, TNF-α, IFN-γ), and an imidazoquinoline immune response modifier (such as but not limited to resiquimod (R848), imiquimod and gardiquimod). More than one second adjuvant may be used.

In one aspect of the foregoing embodiment, the second adjuvant is a TLR agonist.

In one aspect of the foregoing embodiments, the second adjuvant is Monophosphoryl Lipid A (also known as mono-phosphorylated hexaacyl disaccharide and PHAD™) (Avanti Polar Lipids, Alabaster Ala.; catalogue number 699800).

In one aspect of the foregoing embodiments, the second adjuvant is Monophosphoryl 3-Deacyl Lipid A (also known as 3-D PHAD™) (Avanti Polar Lipids, Alabaster Ala.; catalogue number 699852).

In one aspect of the foregoing embodiments, the second adjuvant is a compound as described in US Patent Publication 2015-0110854-A1, which is hereby incorporated by reference for such teachings.

In one aspect of the foregoing embodiments, the additional components of the pharmaceutical composition are fee from compounds that induce an immune response (excluding the synthetic disaccharide lipid compound, antigen and second or additional adjuvant).

The pharmaceutical compositions disclosed may comprise one or more compounds of the present disclosure, alone or in combination with additional active agents, in combination with a pharmaceutically acceptable carrier, excipient or diluent. Examples of such carrier, excipient or diluent and methods of formulation may be found in Remington: The Science and Practice of Pharmacy (20˜″ Ed., Lippincott, Williams & Wilkins, Daniel Limmer, editor). Such pharmaceutical compositions may be used in the manufacture of a medicament for use in the methods of treatment and prevention described herein. The compounds of the disclosure are useful in both free form and in the form of pharmaceutically acceptable salts.

The pharmaceutically acceptable carrier, excipient or diluent described herein, are well-known to those who are skilled in the art. The choice of carrier, excipient or diluent will be determined in part by the particular compound(s), as well as by the particular method used to administer the composition. Accordingly, there is a wide variety of suitable formulations of the pharmaceutical composition of the present invention. The following methods and descriptions are merely exemplary and are in no way limiting. Suitable carriers, excipients or diluents include solvents such as water, alcohol, and propylene glycol, solid absorbants and diluents, surface active agents, suspending agent, tableting binders, lubricants, flavors, and coloring agents. The pharmaceutically acceptable carriers can include polymers and polymer matrices. Typically, the foregoing are chemically inert to the active agents in the composition and has no detrimental side effects or toxicity under the conditions of use. The compounds of the present disclosure and pharmaceutical compositions containing such compounds as described in the instant disclosure can be administered by any conventional method available for use in conjunction with pharmaceuticals, either as individual therapeutic agents or in combination with additional therapeutic agents.

In one embodiment, the compounds of the present disclosure are administered in a therapeutically effective amount, whether alone or as a part of a pharmaceutical composition. The therapeutically effective amount and the dosage administered will, of course, vary depending upon known factors, such as the pharmacodynamic characteristics of the particular agent and its mode and route of administration, the age, health and weight of the recipient; the severity and stage of the disease state or condition; the kind of concurrent treatment; the frequency of treatment; and the effect desired.

The total amount of the compound administered will also be determined by the route, timing and frequency of administration as well as the existence, nature, and extent of any adverse side effects that might accompany the administration of the compound and the desired physiological effect. It will be appreciated by one skilled in the art that various conditions or disease states, in particular chronic conditions or disease states, may require prolonged treatment involving multiple administrations.

The pharmaceutical compositions may be in any form which allows for the composition to be administered to a patient. For example, the composition may be in the form of a solid, liquid or gas (aerosol). Typical routes of administration include, without limitation, oral, topical, parenteral, sublingual and intranasal (e.g., as a spray). The term parenteral as used herein includes iontophoretic, passive transdermal and also subcutaneous injection, intravenous administration, intramuscular injection, intrathecal injection or infusion techniques. The pharmaceutical compositions are formulated to allow the compounds of the present disclosure contained therein to be bioavailable upon administration.

In a one embodiment, the pharmaceutical composition is a stable suspension (such as but not limited to an aqueous suspension) of less than 0.1 μm, 0.2 μm or 0.3 μm and further comprises at least one component selected from the group consisting of phospholipids, fatty acids, surfactants, detergents, saponins, fluorodated lipids, and the like. Formulations suitable for parenteral administration include aqueous and non-aqueous, isotonic sterile injection solutions, which can contain anti-oxidants, buffers, bacteriostats, and solutes that render the formulation isotonic with the blood of the patient, and aqueous and nonaqueous sterile suspensions that can include suspending agents, solubilizers, thickening agents, stabilizers, and preservatives. The compound(s) can be administered in a physiologically acceptable diluent in a pharmaceutically acceptable carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl .beta.-aminopropionates, and 2-alkylimidazoline quaternary ammonium salts, and (e) mixtures thereof.

The parenteral formulations typically contain from about 0.5% to about 50% by weight of the compound(s) in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

In one embodiment, the pharmaceutical composition is formulated in a manner which can be aerosolized for delivery via nasal or pulmonary inhalation. These aerosol formulations can be placed into pressurized acceptable propellants, such as dichlorodifluoromethane, propane and nitrogen. Such aerosol formulations may be administered by metered dose inhalers. They also may be formulated as pharmaceuticals for non-pressured preparations, such as in a nebulizer or an atomizer.

The compound(s) of the present disclosure, alone or in combination with other suitable components, may be administered in an aqueous solution as a nasal or pulmonary spray and may be dispensed in spray form by a variety of methods known to those skilled in the art. Systems for dispensing liquids as a nasal spray are disclosed in U.S. Pat. No. 4,511,069. The formulations may be presented in multi-dose containers, for example in the sealed dispensing system disclosed in U.S. Pat. No. 4,511,069. Additional aerosol delivery forms may include, e.g., compressed air-, jet-, ultrasonic-, and piezoelectric nebulizers, which deliver the active agent dissolved or suspended in a pharmaceutical solvent, e.g., water, ethanol, or a mixture thereof. Nasal and pulmonary solutions of the present invention typically comprise the drug or drugs to be delivered, optionally formulated with a surface-active agent, such as a nonionic surfactant (e.g., polysorbate-80), and one or more buffers. In some embodiments of the present invention, the nasal spray solution further comprises a propellant. The pH of the nasal spray solution is optionally between about pH 3.0 and 6.0, preferably 4.5±0.5. Suitable buffers for use within these compositions are as described above or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, chlorobutanol, benzylalkonimum chloride, and the like. Suitable surfactants include, but are not limited to, oleic acid, sorbitan trioleate, polysorbates, lecithin, phosphatidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

Within alternate embodiments, nasal and pulmonary formulations are administered as dry powder formulations comprising the active agent in a dry, usually lyophilized, form of an appropriate particle size, or within an appropriate particle size range, for intranasal delivery. Minimum particle size appropriate for deposition within the nasal or pulmonary passages is often about 0.5 μm mass median equivalent aerodynamic diameter (MMEAD), commonly about 1 μm MMEAD, and more typically about 2 μm MMEAD. Maximum particle size appropriate for deposition within the nasal passages is often about 10 μm MMEAD, commonly about 8 μm MMEAD, and more typically about 4 μm MMEAD. Intranasally and pulmonaryly respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI), which relies on the patient's breath, upon pulmonary or nasal inhalation, to disperse the power into an aerosolized amount. Alternatively, the dry powder may be administered via air-assisted devices that use an external power source to disperse the powder into an aerosolized amount, e.g., a piston pump.

To formulate compositions for nasal or pulmonary delivery, the active agent can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween SO), solubility enhancing agents (e.g., cyclodextrins and derivatives thereof), stabilizers (e.g., serum albumin), and reducing agents (e.g., glutathione) can be included. When the composition for nasal or pulmonary delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about ⅓ to 3, more typically ½ to 2, and most often ¾ to 1.7.

In one embodiment, a pharmaceutical composition of the disclosure is an emulsion. Single or multiphase emulsion systems are known in the art and may be used. Oil in water emulsion adjuvants and water in oil emulsions may be used. In one embodiment, a pharmaceutical composition of the disclosure is an emulsion of oil in water wherein the synthetic disaccharide lipid compound is incorporated in the oil phase. The oil may be any vegetable oil, fish oil, animal oil or synthetic oil, which is not toxic to the subject and is capable of being transformed by metabolism. Nuts (such as peanut oil), seeds, and grains are common sources of vegetable oils. Squalene is an unsaturated oil which is found in large quantities in shark-liver oil, and in lower quantities in olive oil, wheat germ nil, rice bran oil, and yeast. In one embodiment, the oil in water emulsions are squalene in water emulsions. Such emulsions may contain additional components such as a second adjuvant and other compounds such as antioxidants and other lipid compounds to stabilize the emulsion. The size of the oil droplets found within the stable oil in water emulsion are preferably less than 1 μm, such as in the range of 25 to 500 nm. The methods of producing oil in water emulsions are well known.

The compounds and compositions of the present disclosure can be presented in unit-dose or multi-dose sealed containers, such as ampules and vials, requiring only the addition of the sterile liquid excipient, for example, water for injections, immediately prior to use. Certain compositions can be stored in a freeze-dried (lyophilized) condition if desired. Extemporaneous injection solutions and suspensions can be prepared from sterile powders, granules, and tablets. The requirements for effective pharmaceutically acceptable carriers for injectable compositions are well known to those of ordinary skill in the art. See Pharmaceutical and Pharmacy Practice, J.B. Lippincott Co., Philadelphia, Pa., Banker and Chalmers, Eds., 238-250 (1982) and ASHP Handbook on Injectable Drugs, Toissel, 4th ed., 622-630 (1986).

In one embodiment, the pharmaceutical composition is in the form of a liposome or other slow release mechanism. Suitable slow release mechanisms are described in U.S. Pat. No. 5,888,519 (which is hereby incorporated by reference for such teaching) and include polymers of various types, microcapsules, and microspheres.

Preferred methods for making liposome preparations are described by Bangham (Bangham et. al., 1965). This preparation involves dissolving phospholipids in an organic solvent which is then evaporated to dryness leaving a thin lipid film on the inside of the test tube. The dry lipid film is then hydrated in an appropriate amount of aqueous phase and the mixture is heated to above the phase transition temperature of the lipids and allowed to “swell”. The resulting liposomes which consist of multilamellar vesicles (MLV's) are dispersed by shaking the test tube. The lipids constituting the vesicular bilayer membranes are organized such that the hydrophobic hydrocarbon “tails” are oriented toward the center of the bilayer while the hydrophilic “heads” orient towards the in- and outside aqueous phase, respectively. This preparation provides the basis for producing unilamellar vesicles (UV) by methods such as sonication (Papahadjopoulos et. al., 1967) or extrusion as described by Cullis et. al. in U.S. Pat. No. 5,008,050 (each of the foregoing are hereby incorporated by reference for such teaching).

Liposomes are ordinarily understood to consist of lipid membranes that are capable of enclosing an internal aqueous space and the membranes may consist of a variety of types of lipids. For clarity, the term liposome should not be construed to require the presence of a closed membrane, rather the term should be understood to require that the lipids self-associate such that they form a particulate structure. Among the lipids that have been used either alone or in combination with other lipids to construct liposomes are included phospholipids, glycolipids, glycophospholipids, diglycerides, triglycerides, sterols, steroids, terpenoids, free fatty acids, and lipoidal vitamins.

Release of materials from liposomes most commonly occurs by diffusion but a number of other release mechanisms may also apply. Furthermore, the liposome may act solely as a carrier rather than as a drug release reservoir. The result is a slow release of the compound from the liposome. These mechanisms are described in further detail by in Langer, R., New methods of drug delivery. Science 249:1527-1533 (1990). In a particular embodiment, liposomes for use in the pharmaceutical formulations of the present disclosure comprise a mixture of dimyristoyl phosphatidylcholine (DMPC), dimyristoyl phosphatidylglycerol (DMPG), and cholesterol (Chol) In a particular embodiment, the DMPC/DMPG/Chol are present in molar ratios of 9/1/7. In another particular embodiment, the DMPC/DMPG/Chol are present in molar ratios of 1.8/0.2/1.5. The compounds of the present disclosure may be incorporated into such liposomes as is known in the art (see for example, PCT publication numbers WO2007/068411 and WO2005/081872 which are hereby incorporated by reference herein for such teachings) and described herein.

### EXAMPLES

### Example 1—Purity of Synthetic Disaccharide Lipid Compounds

Synthetic disaccharide lipid compounds of the present disclosure were synthesized and characterized. The compounds synthesized include compounds of the general structural formula V.

### Purity of Compounds Va to Vd

Compound Va has the structure shown below (as well as in general structural formula V). The molecular weight of this compound is 1747.469 (C₉₆H₁₈₄N₃O₂₁P).

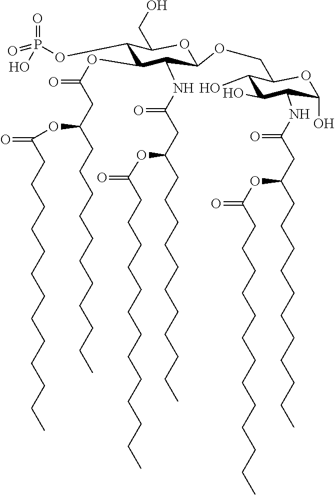

In this compound, acyl chains are present at the 2, 2′ and 3′ positions, with 2 acyl chains being present at the 2, 2′ and 3′ positions. Each acyl chain has a chain length of 14 carbons and each acyl chain is saturated.

The compound was analyzed by TLC, phosphorous NMR, proton NMR and mass spectroscopy. The results are shown in Table 1 below. As can be seen the compound was determined to be over 99% pure and to have a structure consistent with the structure shown above.

| Test | Specification | Result |
|---|---|---|
| Thin-Layer | One spot with iodine | Pass |
| Chromatography | One spot with molybdate | Pass |
| | One spot on charring | Pass |
| HPLC (ELSD) | NLT 95% Purity | 100% Purity |
| | | (AUC) |
| Proton NMR | Consistent with Structure | Consistent with |
| | | Structure |
| Phosphorous NMR | Contains one phosphorous | Pass |
| | shift (ppm) | |
| Mass Spectrometry | [M − NH₄]⁻ = 1729.469 ± 1 amu | Pass |

### PROPHETIC EXAMPLES

One or more of the prophetic examples described below may be performed to analyze the immunostimulatory properties of the molecules of the present disclosure. Similar assays may be substituted for the assays below.

### Prophetic Example 1—Synthetic Disaccharide Lipid Compounds Stimulation of the Human and Mouse TLR-4 Receptor

In this example, the ability of the synthetic disaccharide lipid compounds of the present disclosure will be tested for their ability to activate the human and mouse Toll-like receptor 4 (TLR-4). TLR-4 detects the lipid A portion of lipopolysaccharide (LPS) found in most gram-negative bacteria and is thus important in the activation of the innate immune system. TLR-4 has also been designated as CD284. TLR-4 signals the presence of LPS by associating with two other cell-surface proteins, LY96 (or MD2) and CD14, and when the TLR-4:LY96:CD14 complex binds LPS the intracellular NFκB signaling pathway is activated. Mutations in the TLR4 gene have been associated with differences in LPS responsiveness.

HEK-293 cell lines engineered to functionally over-express human or murine TLR-4 receptor will be utilized in the experiments below. These cells also contain a reporter gene (a secreted alkaline phosphatase) under the control of an NFκB inducible promoter. TLR-4 activation results are given as optical density (OD) values after 18 hours stimulation of the designated HEK-293 cell lines.

Samples and controls will be tested in duplicate on recombinant HEK-293 cell lines. The negative controls for the assay will be the parental HEK-293 cell lines transfected only with the reporter gene. Negative control cells will be stimulated with TNF-α, an inducer of NFκB activity. The positive controls for the assay will be HEK-293 cells transfected with human TLR-4 (hTLR-4) or mouse TLR-4 (mTLR-4) along with the reporter construct and activated with LPS 0111 (obtained from E. coli 0111:B4strain) from 100 ng/ml to 0.01 ng/ml or with LPS K12 (obtained from E. coli K12 strain) from 100 ng/ml to 0.01 ng/ml.

The synthetic disaccharide lipid compound to be used in this assay may be any presented herein, including the compound of the structure Va to Vd. As a comparison, results may also be presented using compounds of the prior art, for example the hexa-acyl disaccharide lipid compound known as PHAD™ (Avanti Polar Lipids, Alabaster, Ala.). PHAD™ has been shown to have immunostimulatory properties. Test compounds will be used at relevant concentrations, for example 10 μg/ml to 0.01 μg/ml when tested alone or at 5 μg/ml to 0.005 μg/ml each when tested in combination.

Positive results indicate the compounds are effective in stimulating human and murine TLR-4, which is indicative of immunostimulatory properties. The compounds of the present disclosure are expected to stimulate human and murine TLR-4.

### Prophetic Example 2—Synthetic Disaccharide Lipid Compounds Stimulate Mouse Splenocyte and B-Cell Proliferation and Increased Viability In Vitro

In this example, the ability of the synthetic disaccharide lipid compounds of the present disclosure will be tested for their ability to stimulate the proliferation and to increase the viability of mouse splenocytes and B-cells in vitro. The synthetic disaccharide lipid compound to be used in this assay may be any presented herein, including the compound of the structure V. As a comparison, results may also be presented using compounds of the prior art, for example the hexa-acyl disaccharide lipid compound known as PHAD™ (Avanti Polar Lipids, Alabaster, Ala.). PHAD™ has been shown to have immunostimulatory properties.

Compounds may be provided in powder form and solubilized in DMSO at suitable concentrations (for example 5 mg/ml), then diluted with sterile pyrogen-free distilled water for a stock solution concentration (for example, 500 μg/ml). These stock solutions will be further diluted in RPMI complete media to make working solutions (for example, 300 μg/ml, 100 μg/ml, and 30 μg/ml), which can then diluted into the appropriate cell culture well at a 1:100 dilution for final test concentrations (for example 3, 1, and 0.3 μg/ml); for combinations the concentrations may be halved. As a solvent control, DMSO will be appropriately diluted in the same media to achieve the required concentration.

Spleens from 6 C57BL/6 mice will be collected and each spleen mechanically dissociated into a single cell suspension with a 70 μm cell strainer. Each single cell suspension of splenocytes will be diluted in complete media (for example RPMI+10% heat-inactivated FBS, 2 mM L-glutamine, 100 U of penicillin/mL, 100 μg of streptomycin/mL and 50 μM 2-Mercaptoethanol), and manual counts of nucleated viable cells (i.e. all cells except red blood cells) will be performed.

B cells will be isolated from the splenocytes of mice using an appropriate method (for example, the EasySep™ Mouse B cell Enrichment Kit, STEMCELL Technologies, Catalog 19754A, Lot 11K41946). To determine the purity after cell separation, cells will be stained with antibodies against an appropriate marker (for example, CD45 using hematopoietic marker; BD Bioscience, Catalog 553080, Lot 49362 and CD19 using B cell marker; eBioscience, Catalog 1209382, Lot E01113-1620), and analyzed by flow cytometry (Accuri® C6 Flow Cytometer) for percentage of markers.

For analysis of proliferation, splenocytes and B cells will be cultured in the presence of the compounds of the present disclosure (including compound V), both alone and in combination with additional lipid A compounds, or the solvent control at 37° C. in 5% CO₂. After 48 hours, detection and quantitation of cell proliferation will be measured using an appropriate assay (for example, the Cell Proliferation ELISA, BrdU, Roche Diagnostics, Catalog 11 647 229 001, Lot 13073200). When BrdU is utilized, after incubation with BrdU, the cell plates will be centrifuged to pellet the cells (plate 1), and the supernatants transferred to a fresh plate (plate 2) also centrifuged to pellet any remaining floating cells. Supernatants from plate 2 will then transferred to a fresh plate and frozen at −20° C. Cell plates 1 and 2 will be used in the proliferation assay to determine the amount of proliferation, as indicated by the absorbance (OD) of each well. The OD values of each sample will be adjusted for non-specific binding by subtracting the OD values of the control wells. OD values from plate 1 and 2 will be added together to give the total proliferation index for each sample.

For analysis of viability, splenocytes and B cells will be cultured in the presence of the compounds of the present disclosure (including compound V), both alone and in combination with additional lipid A compounds, or the solvent control at 37° C. in 5% CO2. After a 72 hour incubation, the cell plates will be centrifuged to pellet the cells and the supernatants transferred to a fresh plate and centrifuged again to pellet any remaining floating cells. The supernatants will be transferred to a fresh plate and frozen at −20° C. for subsequent IgM/IgG evaluation (see below), while the cell pellets from the two plates will be combined and subjected to viability assessment using an appropriate assay (for example, the Cell-Titer Glo, Promega, Catalog G7571/2/3, Lot 9218).

For the cell proliferation and viability assays, cells will be seeded in flat-bottomed 96-well tissue culture-treated plates (Corning® Costar® 3595). Splenocytes will be plated at an appropriate density (for example 1×10⁵ cells/well for the proliferation assay and 1×10⁶ cells/well for the viability assay. B cells will be plated at an appropriate density (for example 1×10⁵ cells/well for both assays). Cell cultures will be monitored on a daily basis for changes in the culture morphology or in the media (e.g., yellowing of the media).

The compounds of the present disclosure are expected to stimulate the proliferation of mouse splenocytes and B cells in vitro and to have no detrimental impact on viability of mouse splenocytes or B cells.

### Prophetic Example 3—Synthetic Disaccharide Lipid Compounds Stimulate IgM and IgG Secretion from Mouse Splenocytes and B-Cells In Vitro

In this example, the ability of the synthetic disaccharide lipid compounds of the present disclosure will be tested for their ability to stimulate IgM and IgG secretion from mouse splenocytes and B-cells in vitro. The synthetic disaccharide lipid compound to be used in this assay may be any presented herein, including the compound of the structure V. As a comparison, results may also be presented using compounds of the prior art, for example the hexa-acyl disaccharide lipid compound known as PHAD™ (Avanti Polar Lipids, Alabaster, Ala.). PHAD™ has been shown to have immunostimulatory properties. Test compounds will be used at relevant concentrations, for example 10 μg/ml to 0.01 μg/ml when tested alone or at 5 μg/ml to 0.005 μg/ml each when tested in combination.

Materials and the conditions for splenocytes and B cell cultures will be as described in Prophetic Example 2. Splenocytes and B cells will be plated at appropriate densities (for example 1×10⁶ cells/well for splenocytes and 1×10⁵ cells/well for B cells).

For assessment of IgM and IgG secretion, splenocytes and B cells will be cultured in the presence of the compounds of the present disclosure either alone or in combination with compounds of the prior art as described above, or the solvent control at 37° C. in 5% CO₂. After a 72 hour incubation, the cell plates will be centrifuged to pellet the cells and the supernatants transferred to a fresh plate and centrifuged again to pellet any remaining floating cells. The supernatants will be transferred to a fresh plate and frozen at −20° C. IgM and IgG will be quantitated by appropriate assay (for example, for IgM the Mouse IgM ELISA Quantitation Set, Bethyl Laboratories, Catalog E90-101, Lot E90-101-25 and for IgG the Mouse IgG ELISA Quantitation Set, Bethyl Laboratories, Catalog E90-131, Lot E90-131-29).

The compounds of the present disclosure are expected to stimulate IgM and/or IgG production.

### Prophetic Example 4—Synthetic Disaccharide Lipid Compounds Stimulate IL-12 Production in Vitro

In this example, the ability of the synthetic disaccharide lipid compounds of the present disclosure will be tested for their ability to stimulate Interleukin (IL)-12 production from a suitable cell line (for example, J774A cells which are murine monocyte/macrophage cells derived a tumor in a female BALB/c mouse) in vitro. IL-12 is an important immune modulator as its production during microbial infections regulates innate responses and determines the type and duration of adaptive immune response. IL-12 induces interferon-γ (IFN-γ) production by natural killer cells, T cells, dendritic cells, and macrophages. IL-12 also promotes the differentiation of naive CD4⁺ T cells into T helper 1 (Th1) cells that in turn produce IFN-γ and aid in cell-mediated immunity. Thus, IL-12 plays a central role in coordinating innate and adaptive immunity and its production is a marker for immunostimulatory properties of a compound.

The synthetic disaccharide lipid compound to be used in this assay may be any presented herein, including the compound of the structure V. As a comparison, results may also be presented using compounds of the prior art, for example the hexa-acyl disaccharide lipid compound known as PHAD™ (Avanti Polar Lipids, Alabaster, Ala.). PHAD™ has been shown to have immunostimulatory properties. Test compounds will be used at relevant concentrations, for example 10 μg/ml to 0.01 μg/ml when tested alone or at 5 μg/ml to 0.005 μg/ml each when tested in combination.

J774A cells will be plated on 24 well plates at an appropriate density (for example, 10⁵ cells/well) and grown under standard cell-culture conditions (for example for 48 hours in DMEM with 10% FBS). Cells will be serum-deprived for 18 h in appropriate media containing the test compounds dissolved in DMSO and incubated an additional 24 hours. The cell media will be harvested and centrifuged (for example at 16,900×g for 10 minutes) and transferred to fresh tubes and frozen at −80° C. until analysis. Mouse IL-12 (p40) will be determined with an appropriate assay (for example the Mouse IL-12 p40 NonAllele-specific Quantikine ELISA (R&D Systems, Minneapolis, Minn.; catalogue No. MP 400).

The compounds of the present disclosure are expected to stimulate IL-12 production.

What is claimed:

1. An essentially pure compound having the structure: 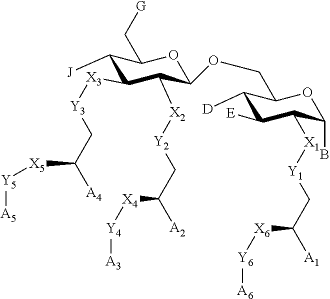
or a pharmaceutically acceptable salt thereof, wherein:
X₁, X₂, X₃, X₄, X₅ and X₆ are each independently absent or selected from —O—, —NH— or —CH₂—;
Y₁, Y₂, Y₃, Y₄, Y₅ and Y₆ are each independently selected from H, —CH₂—, or —C(═O)—, provided that at least 3 of Y₁, Y₂, Y₃, Y₄, Y₅ and Y₆ are a group other than H and further provided that when one of Y₁, Y₂, Y₃, Y₄, Y₅ and Y₆ is H, then the groups which are linked, directly or indirectly, to Y₁, Y₂, Y₃, Y₄, Y₅ and Y₆ are absent;
D, E and G are each independently selected from C₁-C₄ alkyl, —OH, —SH, —OC(═O)(CH₂)ₘ—CH₃, —OC(═O)(CH₂)ₙC(═O)OH, —OC(═O)CH(NH₂)(CH₂)ₙC(═O)OH or —OC(═O)CH₂CH(OH)(CH₂)ₙCH₃;
J and B are each independently selected from OH, OR₁, H, —OP(═O)(OH)₂,
—OP(═O)(OH)(OR₂), —OP(═O)(OR₂)₂, —OS(═O)(OH)₂, —OS(═O)(OR₂)₂, —OS(OH)₂, —OS(OR₂)₂, —C(═O)OH, —C(═O)OR₂ or an acidic group;
A₁, A₂, and A₄ are each independently selected from C₆ to C₂₀ substituted or unsubstituted alkyl or alkenyl;
A₃, A₅ and A₆ are each independently selected from C₈ to C₂₂ substituted or unsubstituted alkyl or alkenyl;
R₁ is a substituted or unsubstituted C₁-C₄ alkyl;
R₂ is independently for each occurrence, H, OH, alkyl, substituted alkyl or N-linked amino acid residue; and
m and n are each independently an integer from 0 to 5.

2. The compound of claim 1, wherein A₁, A₂, and A₄ are each a C₁₁ unsubstituted alkyl, A₃ is a C₁₁ unsubstituted alkyl and A₅ and A₆ are each a C₁₃ unsubstituted alkyl.

3. The compound of claim 1, wherein A₁ A₂, and A₄ are each a C₁₁ unsubstituted alkyl, A₆ is a C₁₅ unsubstituted alkyl and A₅ and A₃ are each a C₁₃ unsubstituted alkyl.

4. The compound of claim 1 having the structure:

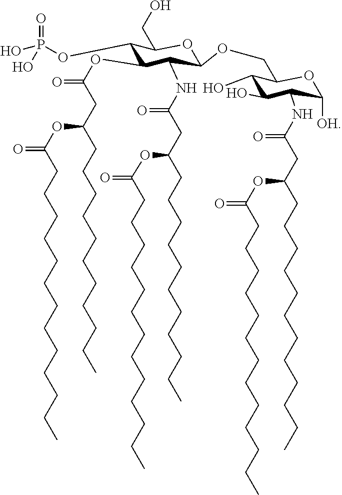

5. The compound of claim 1 having the structure:

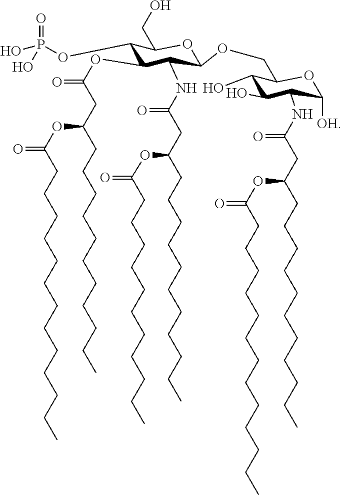

6. The compound of claim 1 having the structure:

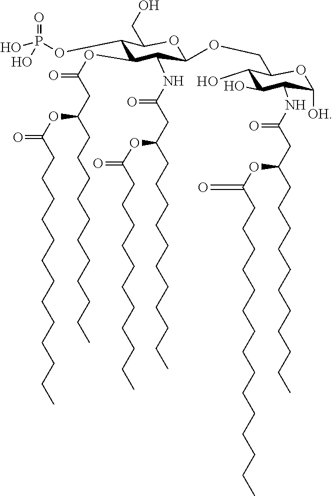

7. The compound of claim 1 having the structure:

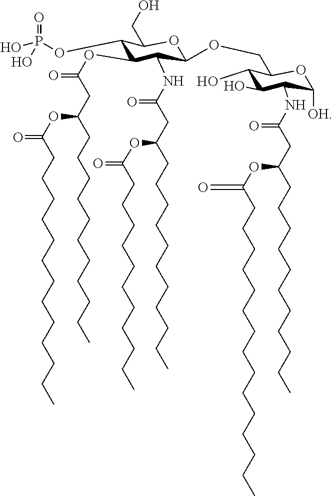

8. The essentially pure compound of claim 1, wherein the compounds is at least 95% pure with respect to the synthetic disaccharide lipid compounds as measured on a weight basis.

9. A pharmaceutical composition comprising:
a. a pharmaceutically acceptable carrier, excipient or diluent;
b. an optional antigen; and
c. an essentially pure compound of claim 1.

10. The pharmaceutical composition of claim 9, wherein the composition comprises an antigen.

11. The pharmaceutical composition of claim 9, wherein the composition comprises more than one antigen.

12. The pharmaceutical composition of claim 9, wherein the composition further comprises a second adjuvant.

13. The pharmaceutical composition of claim 12, wherein the second adjuvant is a mono-phosphorylated hexaacyl disaccharide.

14. The pharmaceutical composition of claim 9, wherein the composition is a vaccine composition.

15. The pharmaceutical composition of claim 9, wherein the composition is useful in stimulating or enhancing an immune response in a subject.

16. The pharmaceutical composition of claim 9, wherein the pharmaceutically acceptable carrier is a liposome.

17. The pharmaceutical composition of claim 9, wherein the compound has the structure:

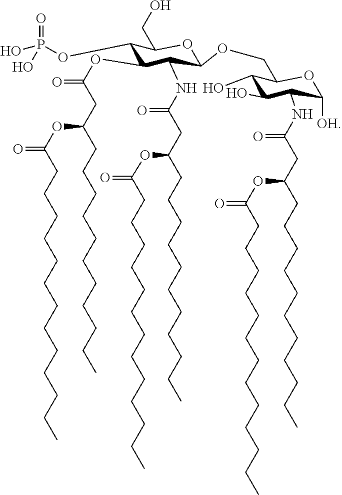

18. The pharmaceutical composition of claim 9, wherein the compound has the structure:

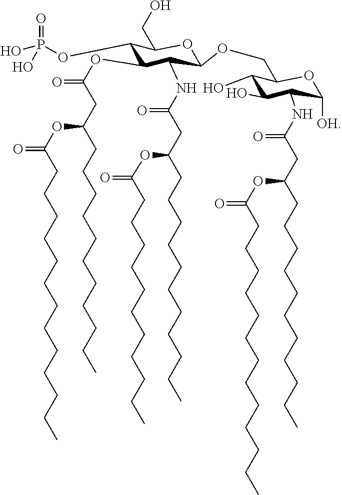

19. The pharmaceutical composition of claim 9, wherein the compound has the structure:

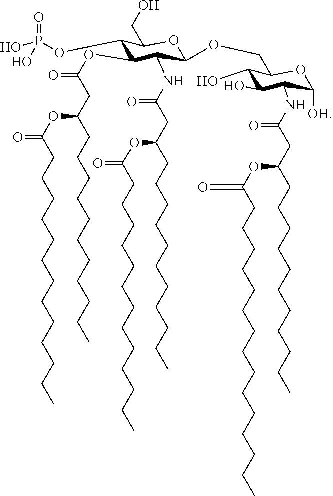

20. The pharmaceutical composition of claim 9, wherein the compound has the structure: [chemical structure]
